(19) United States
(12) Patent Application Publication
(10) Pub. No.: US 2007/0183985 A1
(43) Pub. Date: Aug. 9, 2007

(54) Antibacteric composition to be used as mouthwash for sanitizing the buccal cavity

(75) Inventor: Ettora Tallia, Milano (IT)

Correspondence Address: HEDMAN & COSTIGAN P.C., 1185 AVENUE OF THE AMERICAS, NEW YORK, NY, 10036, US

(73) Assignee: BETAFARMA S.P.A., Cesano Boscone (Milano) (IT)

(21) Appl. No.: 10/562,990

(22) PCT Filed: Jul. 3, 2003
(86) PCT No.: PCT/IT03/00419

§ 371 Date: Mar. 12, 2007

(51) Int. Cl. A61K 8/37; A61K 8/31

(52) U.S. Cl. 424/49

(57) ABSTRACT

Are herein disclosed novel preparations of antibacteric emulsified mouthwash compositions, which are emulsified in a stable manner, and the formula of which comprises particular ‘lipophile compounds”; in these oil compounds, having a high substantiveness for the dentinal and gingival tissue, antiseptic substances, exclusively soluble in an oil phase can be dissolved.

### BACKGROUND OF THE INVENTION

The present invention relates to an antibacteric composition, which can be specifically used as a mouthwash composition for sanitizing the buccal cavity.

As is known, an unsuitable and continued poor hygiene of the buccal cavity, represents, according to recent odontological and biologic studies, the primary cause of different pathologies of the dental apparatus and related mucogingival districts.

Paraodontoid diseases, ulcerative stomatites, tissue necroses, and halitoses of the buccal cavity depend, in most cases, on bacteric and fungin infections, persisting from long time periods on the tooth surfaces and adjoining tissues, probably never processed and hindered in their development in a suitable manner.

Also known is the fact that at present, among several sanitary compositions specifically designed for a prophylactic treatment of teeth, gums, buccal mucouses very important are the so-called antibacteric mouthwashes.

These preparations are conventionally constituted by aromatized aqueous solutions, containing hydrosoluble microbiocide principles, which are used for performing cleansing and refreshing rinsing operations in the buccal cavity.

However, microbiocide properties of the above mentioned mouthwash compositions, in a lot of cases, have not been found as efficient, since the contacting time between the dental apparatus and the mouthwash composition is of a short duration.

Moreover, prior mouthwash compositions, exclusively including hydrosoluble antiseptic substances, are removed, by a quick rinsing operation, after their use.

### SUMMARY OF THE INVENTION

Accordingly, the aim of the present invention is to provide such a mouthwash composition, for sanitizing the buccal cavity, and which is specifically designed to hinder the development of bacteria, while optimally removing bacteria from the tooth solid surfaces and periodontal mucous tissues, thereby overcoming the drawbacks affecting prior art mouthwash compositions.

Within the scope of the above mentioned aim, a main object of the present invention is to provide such a mouthwash composition which is specifically adapted to extend in the time its hygienizing activity.

According to the present invention, novel stabile emulsified antibacteric mouthwash compositions are herein disclosed, which mouthwash compositions comprise specifically designed “lipophile compounds”.

In the above oil compounds, having a high substantiveness for the dentinal and gingival tissue, antiseptic substances, exclusively soluble in an oil phase, can be dissolved.

Thus, by using the above mentioned composition, it is possible to form, through the overall buccal cavity and, more specifically, on the teeth, an oil film, which cannot be easily removed by rinsing with water, which film is adapted to extend in the time the hygienizing activity of the composition.

Moreover, the provision, in each individual composition or preparation, of two functional microbiocide substances, dispersed in different phases (i.e. an aqueous and an oil phase) can synergically enhance the individual biologic properties of said substances.

### EXAMPLE

A mouthwash composition, in the form of a stabile emulsified opaque liquid, in which the two component phases, i.e. the oil phase and the aqueous phase, were respectively added with oil-soluble and hydrosoluble antiseptic substances, has been prepared.

In particular, the emulsion composition can be based on different ratios of the two aqueous and oil phases.

In particular, the aqueous phase can vary from about 60% w/w to about 95% w/w.

In addition to the main component, i.e. water, the above mentioned aqueous phase can be integrated by other hydrosoluble substances, such as moistening substances (glycerol, sorbitol, xylitol, glycol), alcohols (ethyl, propylalcohols), fluorinated salts, sweetening agents (saccharine, aspartames) coloring substances, pH adjusting agents and so on.

In such a phase are always present an emulsifying system of oil in water (O/W) type, as well as a water soluble antibacteric substance, suitable to form stable emulsions.

The oil phase can vary from a minimum of about 5% w/w to a maximum of about 40% w/w.

Said oil phase can comprise vegetable oils, mineral oils, aliphatic esters, aliphatic ethers, aliphatic alcohols, tryglicerides and aliphatic hydrocarbons.

The mouthwash composition according to the present invention can further comprise aromatizing substances and oil-soluble antibacteric substances.

It has been found from a lot of practical experiments and tests, that the mouthwash composition according to the present invention is adapted to properly sanitizing the buccal cavity, while hindering the development or growth of and optimally removing bacteria from the solid dental surfaces and periodontal mucous tissues.

1. An antibacteric composition, particularly designed to be used as a mouthwash composition for sanitizing the buccal cavity, characterized in that said antibacteric composition comprises at least an oil phase in which antiseptic substances are dissolved.

2. An antibacteric composition, according to claim 1, characterized in that said antibacteric composition further comprises an aqueous phase, in which water soluble antibacteric substances are dissolved.

3. A composition, according to claim 1 or 2, characterized in that said aqueous phase varies from about 60% w/w to about 95% w/w.

4. A composition, according to one or more of the preceding claims, characterized in that said composition comprises water soluble substances, such as moistening agents, alcohols, fluorinated salts, sweetening substances, coloring substances, pH adjusters and so on.

5. A composition, according to one or more of the preceding claims, characterized in that said moistening substances are selected from glycerol, sorbitol, xylitol, glycoles, the alcohols being selected from ethyl alcohol and propyl alcohol, and said sweetening agents being selected from saccharine and aspartames.

6. A composition, according to one or more of the preceding claims, characterized in that said oil phase varies from about 5% w/w to about 40% w/w.

7. A composition, according to one or more of the preceding claims, characterized in that said oil phase comprises vegetable oils, mineral oils, aliphatic esters, aliphatic ethers, aliphatic alcohols, triglycerides and aliphatic hydrocarbons.

8. A composition, according to one or more of the preceding claims, characterized in that said composition comprises aromatizing oils and substances.

9. A composition, according to one or more of the preceding claims, characterized in that said composition further comprises an emulsifying system of an oil in water (O/W) type, adapted to form stable emulsions.

10. A composition, according to one or more of the preceding claims, characterized in that said composition comprises one or more of the disclosed and/or illustrated characteristics.
